(19) United States
(12) Patent Application Publication
(10) Pub. No.: US 2025/0072946 A1
(43) Pub. Date: Mar. 6, 2025

(54) ORTHOPEDIC FIXATION DEVICE WITH CAPTURED BONE ANCHOR BLOCKING CAM AND METHODS

(71) Applicants: Jason Blain, Encinitas, CA (US); Samuel Tolpen, San Diego, CA (US); Dean Johnson, Idyllwild, CA (US); Greg Martin, Carlsbad, CA (US)

(72) Inventors: Jason Blain, Encinitas, CA (US); Samuel Tolpen, San Diego, CA (US); Dean Johnson, Idyllwild, CA (US); Greg Martin, Carlsbad, CA (US)

(21) Appl. No.: 18/824,835

(22) Filed: Sep. 4, 2024

Related U.S. Application Data
(60) Provisional application No. 63/536,652, filed on Sep. 5, 2023.

(51) Int. Cl. A61B 17/86; A61F 2/30; A61F 2/44

(52) U.S. Cl. CPC A61B 17/8605; A61F 2/30749; A61F 2/4455

(57) ABSTRACT

An orthopedic fixation device operable for securing bone parts comprising one or more blocking cams integrated into a structural support device such as an intervertebral spacer or a fixation plate. The blocking cam is irremovable from the structural support device without destruction of the blocking cam or the structural support device or both. In preferred embodiments, the blocking cam comprises a central body with one or more tabs extending radially from the central body. The blocking cam is rotatable between a locked mode wherein one of the tabs obstructs removal of a bone anchor from a fixator port of the structural support device and an unlocked mode wherein the bone anchor is unobstructed from removal from a fixator port.

### CROSS REFERENCE TO RELATED APPLICATIONS

This application claims priority to Provisional Patent Application No. 63/536,652 filed Sep. 5, 2023, the entire disclosure of which is hereby incorporated by reference and relied upon.

### BACKGROUND OF THE INVENTION

### Field of the Invention

The invention relates generally to orthopedic implants, and more particularly to orthopedic implants with integrated blocking mechanisms used to prevent the back out of bone anchors. The blocking mechanisms are inseparable from the implant.

### Description of Related Art

Prior designs include multi-component blocking devices affixed to the outside or exterior of bone plates or interbody spacers or are contained within the bone-engagement device. These prior devices commonly have protruding geometry and components that accomplish screw/anchor blocking, but leave a prominence that can disrupt soft tissue structures of the body. In addition, in some cases components associated with these blocking assemblies can release from the implant and cause harm to soft tissue structures nearby. Further, devices with integrated blocking mechanisms utilize individual components that are manufactured separately on different machines. This typically requires an array post-manufacturing processes and inspection of the individual parts and tolerances that significantly increases the cost of the implant. These individual parts then need to be assembled. This assembly step further increases the overall cost of the implant and potentially makes the implant cost prohibitive for some users.

What is needed are orthopedic fixation devices with bone anchor locking mechanisms that comprise a smooth outer surface on the orthopedic fixation device for the safety of soft tissue structures of the body. Also needed are orthopedic fixation devices with bone anchor locking mechanisms that are absent of an assembly requirement to reduce overall costs and consequently provide these implants to lower income recipients. What is needed are orthopedic fixation devices with bone anchor locking mechanisms that are wholly captured within the associated bone plate and/or intervertebral implant to eliminate the potential for unintended release of small parts next to blood vessels, nerves, or other soft tissues where they can cause injury.

### SUMMARY OF THE INVENTION

An implantable orthopedic fixation device for supporting bony structures comprising one or more fixator ports for seating a bone anchor therein. The orthopedic fixation device comprising one or more fixator ports and an integrated blocking cam that blocks the fixator port passageway when activated thereby preventing back out of the bone anchor once the bone anchor is advanced and seated in bone.

In one form, the orthopedic fixation device is in the form of a fixation plate, otherwise known as a bone plate.

In one form, the orthopedic fixation device is in the form of an intervertebral spacer operable for placement in the intervertebral space between two vertebral bodies when at least a portion of an intervertebral disc is removed.

In one form, the orthopedic fixation device is used adjacent to a human or other animal skeleton such as a human spine.

In one form, the intervertebral spacer is defined by a boundary wall defining the external boundaries of the intervertebral spacer. The intervertebral spacer is bounded superiorly by a superior face, inferiorly by an inferior face, and radially by an external face.

In one form, the intervertebral spacer comprises a first lateral fixator port, a second lateral fixator port, and a central fixator port whereas each fixator port is configured to house a bone anchor.

In one form, the fixator ports extend from an external face of an intervertebral spacer to either a bone facing inferior face or bone facing superior face which are positioned adjacent to bone in an implanted mode.

In one form, the spacer body comprises a plurality of bone teeth extending from the superior face and inferior face.

In one form, the superior face and inferior face are substantially convex. In some cases, the convexity is substantially the same as the end plate concavity of a typical human spine where the intervertebral spacer will implanted.

In one form, the intervertebral spacer comprises one or more bone cavities extending between the inferior face and superior face. The bone cavities are inset from the boundary wall and at least partially define internal faces of the boundary wall.

In one form, the integrated blocking mechanism is in the form of a blocking cam.

In one form, the blocking cam comprises a rounded central body.

In one form, the blocking cam comprises tabs (first tab, second tab) extending from the central body which switch from an unlocked mode to a locked mode when the central body is rotated. In the locked mode, the tabs interfere with the bone-engagement passageway (fixator ports) and prevent component disassociation.

In one form, the tabs extend through cam windows into fixator ports in a locked mode wherein the bone anchors are prevented from backout.

In one form, the tabs are retracted from the fixator ports into cam windows in an unlocked mode wherein the bone anchors can then be driven into bone or retracted from bone.

In one form, the integrated blocking cam is captured within a cam port in the structural body of the orthopedic fixation device.

In one form, the integrated blocking cam is irremovable from the structural body of the fixation plate or intervertebral spacer upon completion of manufacturing.

In one form, the structural body of the orthopedic fixation device is inserted into an intervertebral/disc space of a human spine.

In one form, the structural body of the orthopedic fixation device is positioned on the surface of bone structures.

In one form, the orthopedic fixation device comprises one or more bone anchors configured for fixation within a bone.

In one form, the bone anchors comprise bone screw threads whereby the bone anchor is advanced by rotation along its central axis for fixation into bone.

In one form, the bone anchors are of an impacted form whereby the bone anchor is advanced by impacting along its central axis with or without rotation.

In one form, the bone anchors are impacted then rotated for the engagement of teeth within the bone.

In one form, the bone anchors are advanced through the fixator ports and advanced into the bone of a recipient.

In one form, after advancing the bone anchor with the blocking cam in an unlocked mode and consequently unobstructing the fixator port, the blocking cam is then rotated to a locked mode such that the cam tabs (first tab, second tab) on the blocking cam interfere with the fixator ports. The locked mode prevents the bone anchor from backing out of the fixator ports thereby preventing or limiting undesired bone anchor disassociation or backout.

In one form, the blocking cam and/or orthopedic fixation device comprises positional lock features that determine a locked and unlocked mode such as complementing detents and dimples or ramps on the surfaces of the blocking cam and/or orthopedic device.

In one form, the positional lock feature on the blocking cam and/or elsewhere on the orthopedic fixation device provide the user a tactile response to indicate advancement to a locked or an unlocked mode.

In one form, the orthopedic fixation device is in the form of an intervertebral spacer with blocking cam for use in one or more of the lumbar, thoracic, and cervical spine.

In one form, the orthopedic fixation device is implanted utilizing an anterior surgical approach of the spine.

In one form, the orthopedic fixation device is implanted utilizing a lateral surgical approach of the spine.

In one form, the orthopedic fixation device is implanted utilizing a posterior surgical approach of the spine.

In one form, the orthopedic fixation device is in the form of an intervertebral spacer and is offered in a variety of sizes to accommodate the boney dimensions and intervertebral spacing of the recipient.

In one form, the orthopedic fixation device is in the form of a bone plate and is offered in a variety of sizes to accommodate the boney dimensions of the recipient.

In one form, the orthopedic fixation device is utilized to stabilize a human spine.

In one form, the orthopedic fixation device is utilized to stabilize an animal spine.

In one form, the intervertebral spacer or bone plate in an orthopedic fixation device houses the blocking cam whereby the locking cam is irremovable from the intervertebral spacer or bone plate after manufacture.

In one form, removal of the blocking cam from the intervertebral spacer or bone plate would destroy one of these components.

In one form, the blocking cam is housed in a cam port situated within the respective intervertebral spacer or bone plate.

In one form, the orthopedic fixation device is constructed utilizing an additive manufacturing process.

In one form, using additive manufacturing, post-production assembly of the blocking cam within the orthopedic fixation device is unnecessary.

In one form, the orthopedic fixation device comprises a smooth outer profile that faces anatomical structures of the recipient upon implantation.

In one form, the orthopedic fixation device comprises a fixation plate for fixation over the surface of bone.

In one form, the fixation plate comprises a plurality of fixator ports that extend through the fixation plate.

In one form, bone anchors extend through the fixator ports to secure the fixation plate to bone.

In one form, a blocking cam is inseparable from the fixation plate and is switchable between a locked mode and an unlocked mode wherein in a locked mode, a tab on the blocking cam blocks removal of an enlarged head of the bone anchor, and wherein in an unlocked mode the bone anchor is unblocked from removal from the fixation plate.

In one form, the blocking cam is manufactured, without assembly within the orthopedic fixation device.

In one form, the blocking cam has an anchor portion that is larger than a restraint portion in a structural member where it resides thereby preventing separation between the blocking cam and structural member.

### BRIEF DESCRIPTION OF THE SEVERAL VIEWS OF THE DRAWINGS

These and other features and advantages of the present invention will become more readily appreciated when considered in connection with the following detailed description and appended drawings, wherein each drawing is according to one or more embodiments shown and described herein, and wherein:

FIG. 1 depicts a top perspective view of an orthopedic fixation device with blocking cams in an unlocked mode;

FIG. 2 depicts a top perspective view of an orthopedic fixation device with blocking cams in a locked mode;

FIG. 3 depicts a bottom perspective view of an orthopedic fixation device;

FIG. 4 depicts a top perspective view of an orthopedic fixation device;

FIG. 5 depicts a top perspective view of an intervertebral spacer with integrated blocking cams;

FIG. 6 depicts an anterior view of an intervertebral spacer with integrated blocking cams;

FIG. 7 depicts a front cross-sectional view of an intervertebral spacer with integrated blocking cams through plane C in an unlocked mode;

FIG. 8 depicts a front cross-sectional view of an intervertebral spacer with integrated blocking cams through place C with one blocking cam in a locked mode and one in an unlocked mode;

FIG. 9 depicts a side cross-sectional view through plane A of an intervertebral spacer with integrated blocking cam with a view through a cam window;

FIG. 10 depicts a cross-sectional view through plane B of an intervertebral spacer with integrated blocking cams;

FIG. 11 depicts an opposed side cross-sectional view through plane A of an intervertebral spacer with integrated blocking cam;

FIG. 12 depicts a cross-sectional view through plane B of an intervertebral spacer with blocking cams removed;

FIG. 13 depicts an opposed cross-sectional view through plane B of an intervertebral spacer with blocking cams removed;

FIG. 14 depicts a top perspective view of an intervertebral spacer having an open lattice structure;

FIG. 15 depicts a central cross-sectional view of the intervertebral spacer of FIG. 14 highlighting the open lattice construction;

FIG. 16 depicts a top perspective view of a bone anchor;

FIG. 17 depicts a side perspective view of the bone anchor of FIG. 16;

FIG. 18 depicts a cross-sectional view of the bone anchor of FIG. 16 along a central plane;

FIG. 19 depicts a posterior perspective view of a pair of blocking cams;

FIG. 20 depicts an anterior perspective view of the pair of blocking cams of FIG. 19;

FIG. 21 depicts an anterior perspective view of an orthopedic fixation device inserted in an interverbal space comprising bone anchors and integrated blocking cams;

FIG. 22 depicts an anterior perspective closeup view of the human spine in FIG. 21 with the orthopedic fixation device implanted;

FIG. 23 depicts a superior perspective view of an orthopedic fixation device having a structural member in the form of a fixation plate secured to bone using bone anchors. The orthopedic fixation device includes a captured bone anchor blocking cam;

FIG. 24 depicts a superior perspective view of the orthopedic fixation device of FIG. 23;

FIG. 25 depicts a superior perspective view of the fixation plate as used in the orthopedic fixation device of FIG. 23;

FIG. 26 depicts an opposed perspective view of the fixation plate as used in the orthopedic fixation device of FIG. 23;

FIG. 27 depicts a perspective view of a blocking cam as used in the orthopedic fixation device of FIG. 23;

FIG. 28 depicts an opposed perspective view of a blocking cam as used in the orthopedic fixation device of FIG. 23;

FIG. 29 depicts a cross-sectional view of the fixation plate through plane A as used in the orthopedic fixation device of FIG. 23;

FIG. 30 depicts a close-up cross-sectional view of the fixation plate as used in the orthopedic fixation device of FIG. 23;

FIG. 31 depicts a close-up cross-sectional view of the fixation plate with integrated blocking cam as used in the orthopedic fixation device of FIG. 23;

FIG. 32 depicts a perspective view of a bone anchor as used in the orthopedic fixation device of FIG. 23;

FIG. 33 depicts a side view of a bone anchor as used in the orthopedic fixation device of FIG. 23;

FIG. 34 depicts a cross-sectional view of a bone anchor as used in the orthopedic fixation device of FIG. 23.

### DETAILED DESCRIPTION OF SELECTED EMBODIMENTS OF THE INVENTION

Select embodiments of the invention will now be described with reference to the Figures. Like numerals indicate like or corresponding elements throughout the several views and wherein various embodiments are separated by letters (i.e. 100, 100B, 100C). The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes, or which is essential to practicing the invention described herein.

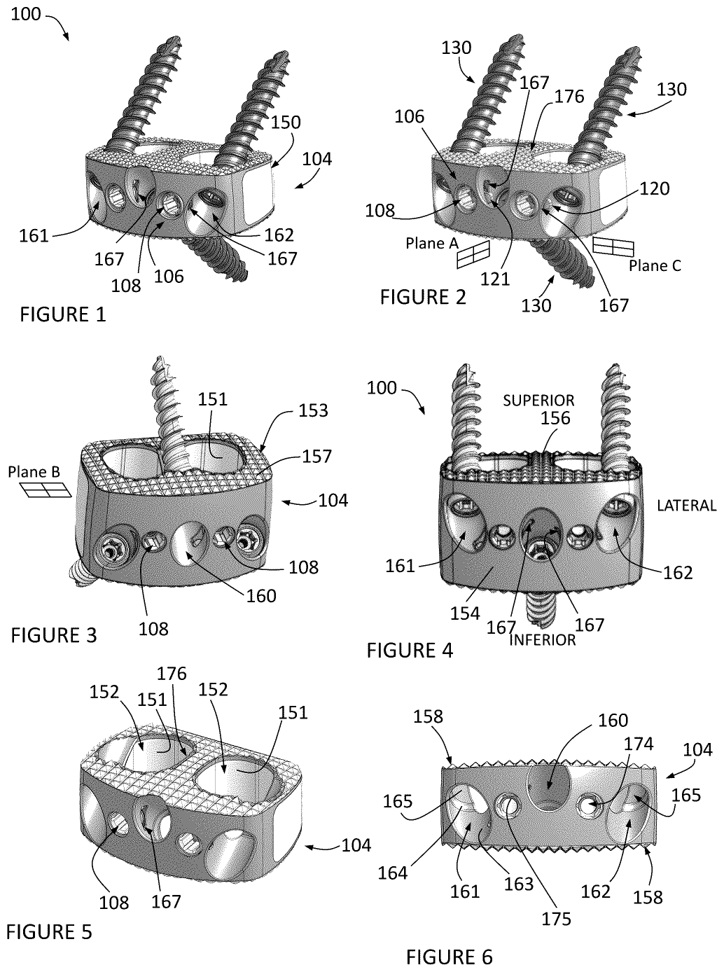

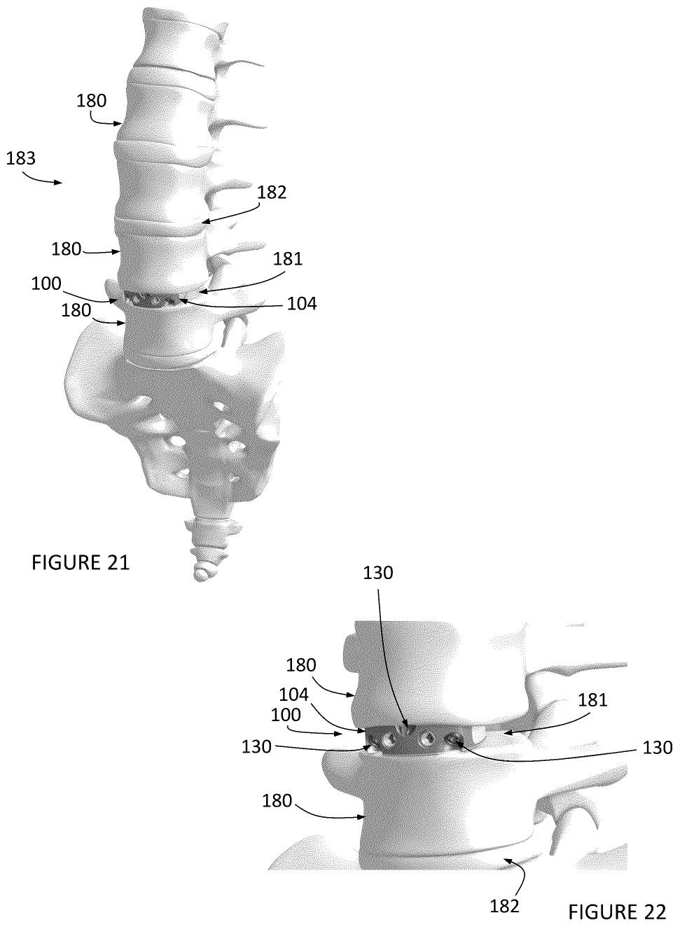

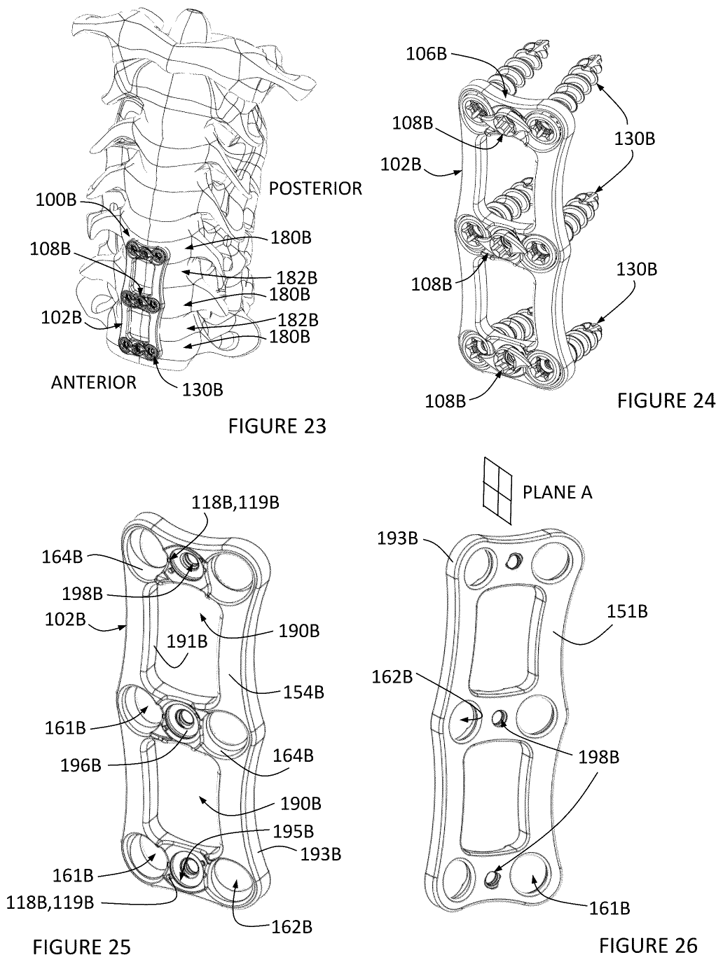

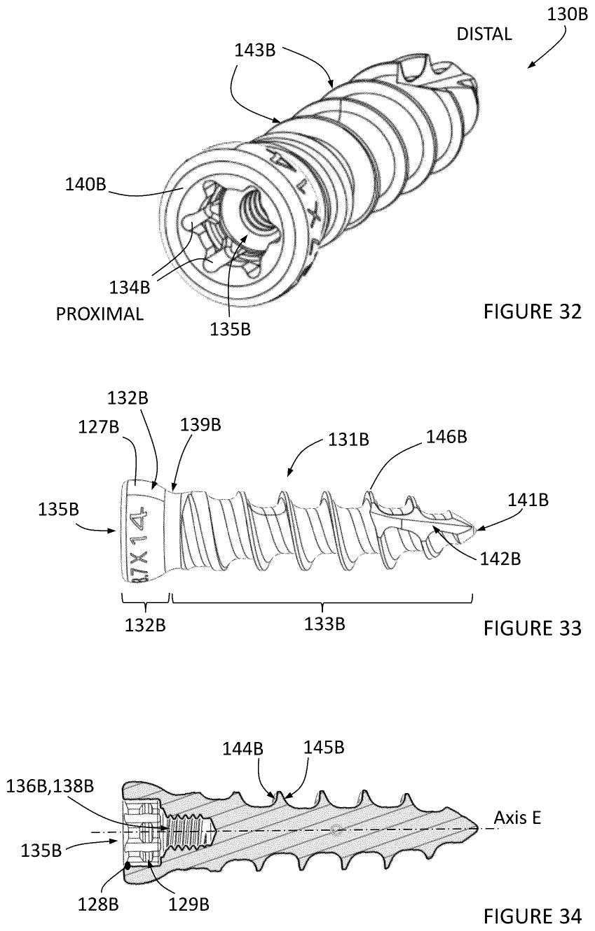

Described below are select embodiments of an implantable orthopedic fixation device for supporting bony structures. The orthopedic fixation devices comprise one or more fixator ports for seating a bone anchor therein and further comprise an integrated blocking cam that blocks the passageway when activated to a locked mode thereby preventing back out of the bone anchor once the bone anchor is advanced and seated in adjacent bone. The orthopedic fixation device can have a structural member in the form of a fixation plate 102B as depicted in FIGS. 23-34, otherwise known as a bone plate. The orthopedic fixation device can also take other forms such as depicted in FIGS. 1-22, where the structural member is in the form of an intervertebral spacer 104 operable for fixation in the intervertebral space 181 between two vertebral bodies 180 when at least a portion of an intervertebral disc 182 is removed. FIG. 21-22 depict the orthopedic fixation device 100 in an implanted mode wherein bone anchors 130 extend through the spacer body 150 into adjacent vertebrae.

Intervertebral spacer 104 is defined by a boundary wall 153 encircling the intervertebral spacer bounded on a superior end by a superior face 156, inferiorly by an inferior face 157, and radially by an external face 154. The intervertebral spacer 104 can also include a dividing wall 176 also extending between the inferior face and superior face and merging with the boundary wall 153. An internal face 151, spaced inward from external face 154 on the boundary wall and on the dividing wall, defines one or more bone cavities 152 for housing bone or bone substitute during the fusion process. Dividing wall 176 is positioned to provide additional support between the vertebral end plate above and the vertebral end plate below and consequently divides the bone cavities into additional bone cavities. As noted in the Figures, the superior face 156 and inferior faces 157 are preferably substantially convex. In preferred embodiments, extending from the inferior face and superior face are a plurality of bone teeth 158. The bone teeth are typically pointed and/or ridged to engage the adjacent end plates and thereby minimized movement therebetween.

Intervertebral spacer 104 comprises a first lateral fixator port 161 offset laterally on one side of the intervertebral spacer, a second lateral fixator port 162 offset laterally on the opposite side, and a central fixator port 160 generally centered on the spacer. The first and second lateral fixator ports are directed from the external face 154 upwards through the bone facing superior face 156, whereas the central fixator port is directed downwards through the bone facing inferior face 157. It is recognized that these directions can be reversed in alternative embodiments. Each fixator port is configured to house a bone anchor for engaging the adjacent intervertebral endplate when implanted. The fixator ports are substantially cylindrical. The first part of the fixator ports are defined by tunnel face 163 which extends from external face 154 having a diameter sufficient to pass the enlarged head 132 of the bone anchor 130. The fixator port then narrows as defined by neck face 164 that is sufficiently small in diameter to offer a seat for enlarged head 132 yet is sufficiently large in diameter to pass shank 133 of bone anchor 130. The final section of the fixator port is defined by shank face 165 which can be sufficient in diameter to pass the shank 133 of bone anchor 130 while simultaneously limiting off axis movement, or have more substantial diameter to provide some level of off axis movement (note FIG. 9 whereby the diameter increases near the bone engaging surface). As noted in the Figures, a portion of the fixator ports defined by shank face 165 can open to the bone cavities 152 as well as to superior face 156 and inferior face 157. In preferred embodiments, tunnel face 163, neck face 164, and shank face 165 are depicted as continuous surfaces along axis F, however, in alternative embodiments, these faces can be interrupted. For example, the neck face can comprise three high points to sufficiently cradle the enlarged head of the bone anchor. Axis E of a bone anchor and axis F of a fixator port can be fully aligned or can be misaligned to a degree in the implanted mode.

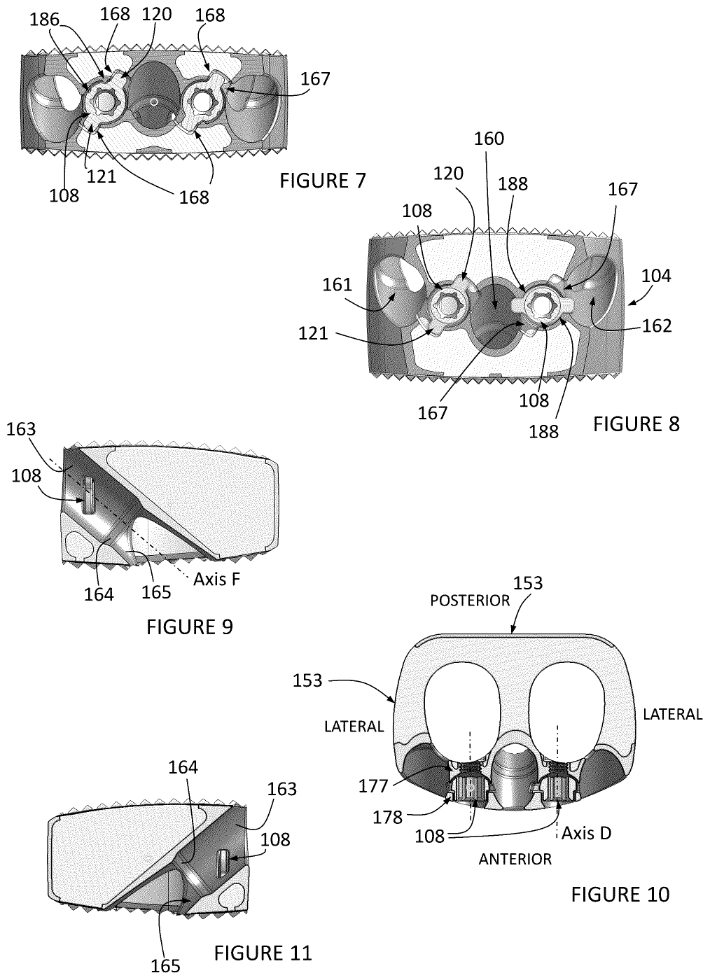

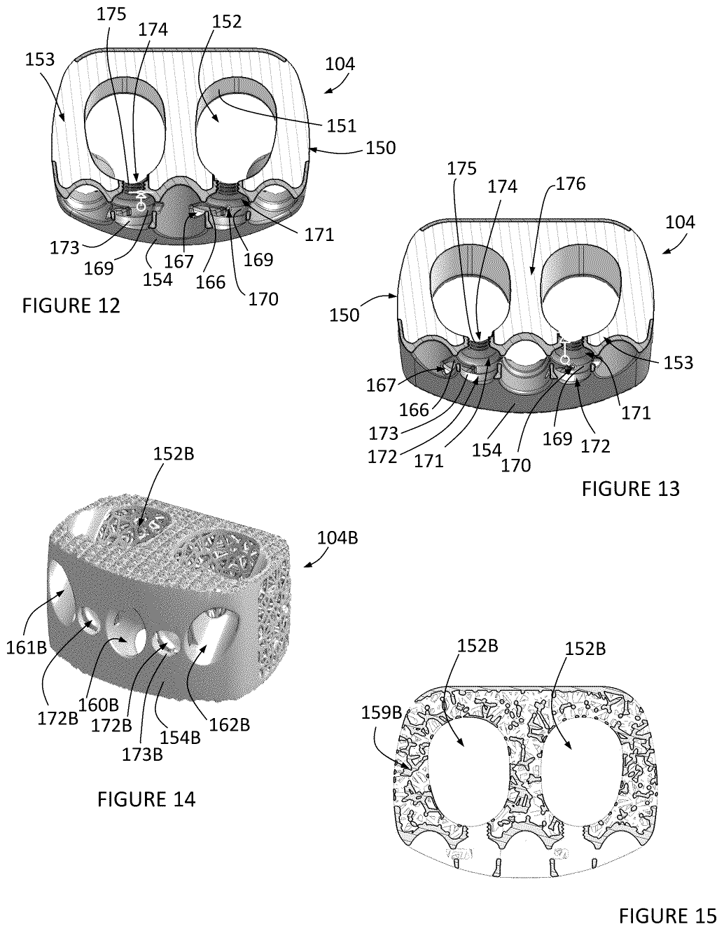

The blocking cams 108 are housed within cam ports 172 (FIGS. 12-14) within boundary wall 153 of spacer body 150 or within a bone plate along a common axis D. In the case of a bone plate, the boundary wall is the form of a straight or curvilinear strut wherein the internal face 151 is adjacent bone in an implanted mode. The central portion of the cam ports 172 are initially defined by cam port face 173 which is substantially cylindrical upon entry at external face 154. Cam port face 173 then steps to head capture face 169 having initially a larger diameter than cam port face 173 before narrowing at pivot seat 171 until intersecting threaded control face 175 which defines control passage 174. Control passage 174 opens into bone cavity 152 through internal face 151 (or to the bone facing side of the plate in the case of a bone plate). Tab guide face 166 extends from the cam port 172 to adjacent fixator ports to define cam windows 167 in which the respective first tab 120 or second tab 121 rotate and extend through to create a retractable barrier in a respective fixator port. In this case, cam window 167 has a substantially rectangular profile but can vary as needed to complement the tabs. As noted in FIG. 7, a portion of the cam window 167 can comprise a cam block 168 to limit rotation of the first tab 120 and/or second tab 121. The enlarged head capture face 169 (FIG. 12-13) is formed in the cam port 172 for housing the enlarged head defined by head face 117 of blocking cam 108. Again, in this embodiment, head capture face 169 maintains a diameter larger than that defined by cam port face 173 thereby preventing escape of the blocking cam. As depicted in FIG. 10, a narrowing pivot seat 171 and control passage 174 creates an inner capture region 177, while cam port face 173 creates an outer capture region 178, between which the blocking cam 108 is unable to escape. However, in alternative embodiments, one or both of the narrowed inner capture region and outer capture regions are absent. In that case, first tab 120 and second tab 121 captured within their respective cam windows 167 solely or in cooperation with a capture region make the blocking cam remain irremovable from boundary wall 153.

A head dimple 170 can be present protruding from cam port 172 for tactile sensation when rotating the blocking cam 108. In some embodiments, the surfaces of head capture face 169 and/or head face 117 are varied (i.e. cammed) such that there is increased friction in predetermined rotational positions of the blocking cam. For example, friction fit locking 188 is depicted in FIG. 8 wherein gaps between the head capture face 169 and head face 117 are absent in the locked mode (right). The user will tactilely feel this greater resistance during locking.

The intervertebral spacer can assume of wide variety of profiles. For example, as depicted in FIG. 1, external face 154 defines a profile that is substantially lima bean shaped or a smaller version of a human endplate. However, the intervertebral spacer can assume other profiles as noted in the prior art such as generally rectangular. As further depicted in the Figures, one of the bone anchors is downward directed and sits midline, whereas two of the bone anchors are upward directed and spaced laterally from the downward directed anchor. This arrangement can be varied without loss of function in alternative embodiments.

The orthopedic fixation device 100 comprises an integrated blocking mechanism 106 for preventing undesired back out of the bone anchors once implanted. In the embodiments depicted, the blocking mechanism 106 is in the form of a blocking cam 108. The blocking cam 108 (FIG. 19-20) comprises a round central body 110 with an internal central face 111 spaced about axis D defining a central drive pocket 113 extending through the central body from a outward facing outward face 114 to and inward facing inward face 115. A plurality of drive faces 112 extend into the central faces for engagement with a camming tool that can impart a rotary force on the blocking cam to switch between a locked mode and unlocked mode. An outer taper 125 can be present between the outward face 114 and central face 111 to ease insertion of a camming tool. Distal to the outward face 114, a pivot face 116 defines a generally cylindrical outer portion of central body 110. Adjacent pivot face 116 is head face 117 which defines a generally cylindrical outer portion of central body 110 having an enlarged diameter compared to pivot face 116. Distally, head face 117 rounds down until terminating at inward face 115. The rounding of head face 117 is configured in size and rounded shape to seat within pivot seat 171 for pivoting therebetween when switching between locked and unlocked modes.

Laterally extending from head face 117 of central body 110 of the blocking cam 108 is a pair of opposed tabs (first tab 120, second tab 121). In this embodiment, the tabs have a substantially square or rectangular profile but can assume other profiles without loss of function. Again, the blocking cam can switch from an unlocked mode to a locked mode when the central body is rotated. In the locked mode, the tabs extend into and interfere with the bone-engagement passageway (fixator ports) and prevent component disassociation such as bone anchor backout. Proximally, first tab 120 and second tab 121 are defined by a substantially flat tab outer face 122 facing outward and by an opposed substantially flat tab inner face 123 facing inward towards the intervertebral spacer. A tab radial face 124, extends between the tab inner face and tab outer face. A detent face 119 is present in this embodiment and defines a dimple, channel, or other depression into the head face. As noted in FIG. 7 a complementing head dimple 186, ridge or boss provide tactile positioning feedback to a user upon rotation of the blocking cam as these complementing features engage and disengage during rotation.

As noted in FIG. 2 and FIG. 8, the first tab 120 and second tabs 121 extend through cam windows 167 into fixator ports in a locked mode. As noted in FIG. 1 and FIG. 7, the first tab 120 and second tabs 121 are retracted into cam windows 167 in an unlocked mode thereby providing full access for insertion and removal of bone anchors 130 through the fixator ports. FIG. 8 depicts blocking cams in both a locked mode (right) and unlocked mode (left). The integrated blocking cam is captured within a cam port in the structural body of the orthopedic fixation device and is thus irremovable from the structural body of the fixation plate or intervertebral spacer upon completion of manufacturing. As noted in the Figures, the diameter of head face 117 exceeds the diameter of the cam port 172 as defined by cam port face 173. In addition, the first tab 120 and second tab 121 are captured within the cam windows 167. Together, these relationships assure the blocking cam cannot escape from the cam port.

Depicted in FIG. 6, the threaded control faces 175 of control passages 174 are clearly accessible through drive pocket 113 due to their smaller diameter. This dimensional relationship facilitates locking an inserter instrument to the control passages by threading to control insertion and positioning of the intervertebral spacer 104. Likewise, drive faces 112 of the blocking cams also remain accessible for switching between locked and unlocked positions by use of a camming tool to impart rotation about axis D.

Depicted in FIGS. 21 and 22, the intervertebral spacer 104 of the orthopedic fixation device 100 is inserted into an intervertebral space 181 between adjacent vertebral bodies 180 of a human spine 183. As shown, the intervertebral spacer 104 of the orthopedic fixation device is positioned on the surface of bone structures which in this case are between the endplates of adjacent vertebrae.

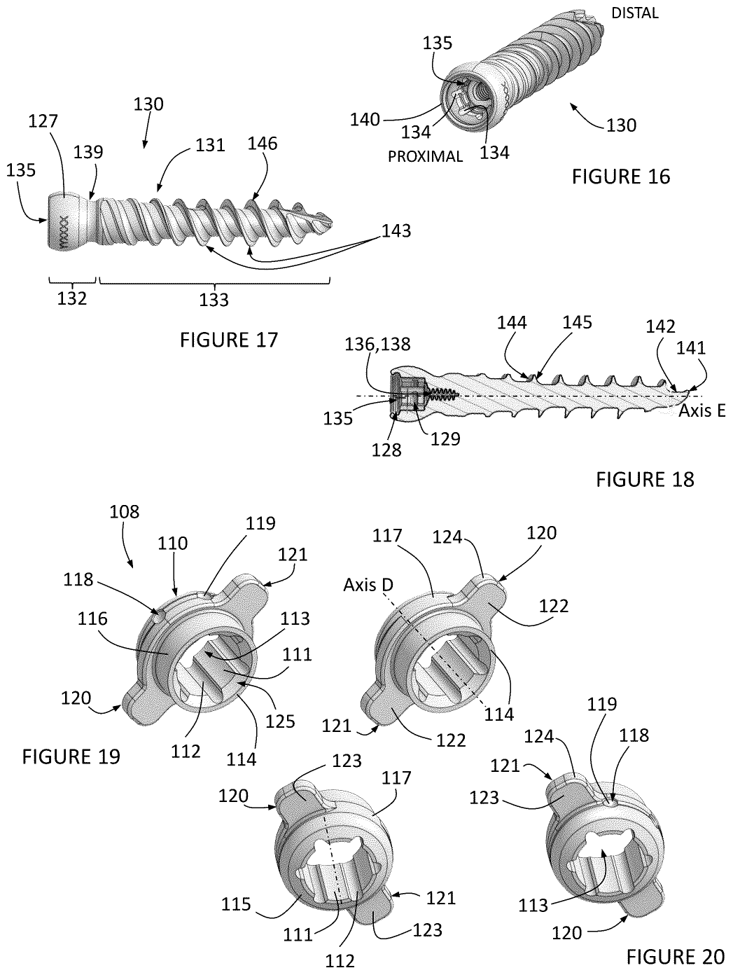

FIGS. 16-18 depict views of one type of bone anchor that can be used with the orthopedic fixation device 100. The bone anchors 130 in this embodiment are bone screws and comprise an elongate screw body 131 that further comprises bone screw threads 143 along the length of the shank 133 of the bone anchor. Note in some bone screws, such as this one, the minor diameter of the screw increases from tip to head. In this embodiment, the bone anchor is advanced by rotation along its central axis E for fixation into bone such as a vertebral body for example. In other embodiments, the bone anchors can be of an impacted form whereby the bone anchor 130 is advanced by impacting along its central axis with or without rotation.

Here, the proximal end of the bone anchor 130 comprises an enlarged head 132 which is at least partially spherical as defined by a head surface 127. A drive recess 135 extends through proximal face 140 into the enlarged head from a proximal end. The drive recess 135 is defined by a plurality of drive surfaces 134 configured for engagement by a drive tool that can impart rotation on the bone anchor 130. Extending radially into drive recess 135 can be one or more annular dents such as depicted as proximal detent 128 and distal detent 129 which can be used for interfacing with tools suited to use this feature. Advancing deeper past drive recess 135, is head lock 136 which is defined by head threads 138. This is yet another feature that can be utilized during surgery for example, for securing the bone screw to an inserter tool. A narrowed neck 139 joins enlarged head 132 with shank 133. At the distal end of bone anchor 130 is distal tip 141 which can be sharpened and in some cases a tip lead 142 is present that leads into the bone screw threads. In the event of a threaded bone anchor, the bone screw threads 143 comprises a proximal thread face 144 facing substantially proximal, and a distal thread face 145 facing substantially distally (towards the distal tip 141). An edge face 146 joins the distal thread face 145 and proximal thread face 144. As noted in FIG. 18 the proximal thread face can be substantially perpendicular to the elongate axis of the bone screw, whereas the distal thread face can be sloped providing the screw more resistance to pullout.

During implantation, the bone anchors are advanced through respective fixator ports and into the bone of a recipient. After advancing the bone anchor with the blocking cam in an unlocked mode and consequently unobstructing the respective fixator port, the blocking cam is then rotated to a locked mode such that the cam tabs (first tab 120, second tab 121) on the blocking cam 108 interfere with the respective fixator port. The locked mode prevents the bone anchor from backing out of the fixator ports thereby preventing or limiting undesired bone anchor disassociation or backout.

The blocking cam and/or orthopedic fixation device can comprise positional lock features that determine a locked and unlocked mode such as complementing detents and dimples or ramps on the surfaces of the blocking cam and/or orthopedic device. Likewise, the positional lock feature on the blocking cam and/or elsewhere on the orthopedic fixation device provide the user a tactile response to indicate advancement to a locked or an unlocked mode. Cam port face 173

As depicted in FIGS. 1 and 3, intervertebral spacer bodies 150 can assume different size profiles and heights depending on the patient needs and can be utilized in the lumbar, thoracic, or cervical spine. A variety of surgical approaches can be used such as anterior, lateral, or posterior. The profile of the intervertebral spacer can change depending on the surgical approach. The orthopedic fixation device depicted in FIG. 1 for example, is suited best for an anterior approach. The intervertebral spacers depicted house the blocking cam whereby the blocking cam is irremovable from the intervertebral spacer or bone plate after manufacture.

As noted in FIG. 1-6, the orthopedic fixation device comprises a smooth external face 154 and outer profile that faces delicate soft tissues and other anatomical structures of the recipient upon implantation. This feature prevents injury to these surrounding tissues.

Elements of the orthopedic fixation devices can be constructed from a variety of implantable materials including polymers such as PEEK, metals such as Ti, and alloys such as Ti-64 and stainless steels. Many of these materials are considered non-porous or low porous. Additive manufacturing can be utilized where needed to create complex or interlocking geometries. The intervertebral spacer can consist of a solid material such as depicted in FIGS. 1-13, or as an open lattice structure 159B as depicted as intervertebral spacer 104B in FIGS. 14-15.

FIG. 23 depicts an orthopedic fixation device 100B comprising a structural member in the form of a fixation plate 102B in an implanted mode. In this case, the fixation plate is in the form of anterior cervical plate used to fixate a human's cervical spine. Like the intervertebral spacer introduced earlier, orthopedic fixation device 100B comprises an integrated blocking mechanism 106B. As noted in FIG. 24, orthopedic fixation device 100B comprises a plurality of bone anchors 130B which in this embodiment are in the form of bone screws as indicated by the bone screw threads 143B which includes a proximal thread face 144B facing proximally and a distal thread face 145B facing distally. The slope of these faces can vary as previously described. Other features can be included as labeled in the figures and are simply different versions of those previously described in bone anchor 130.

Depicted in FIG. 25 is one embodiment of fixation plate 102B which comprises an outward facing external face 154B and an inward facing internal face 151B which is typically placed against the bone when implanted. Note that the internal face can be curved, for example, it can be concave to generally match the natural concavity of the bone in which it is utilized. A bounding face 193B encircles the fixation plate extending between the external face and internal face. In some fixation plate embodiments, one or more plate windows 190B will extend through the plate. The plate windows are defined by a window face 191B that extends between the external face and internal face. In some embodiments, the plate windows are substantially rectangular, but they can assume other profiles.

Fixation plate 102B will have a plurality of fixator ports that will be used to house bone anchors 130B for securing the plate against the bone. For example, fixation plate 102B can be used for cervical spine fixation. The fixator ports are provided in pairs including a first lateral fixator port 161B opposite a second lateral fixator port 162B generally mirrored across mid-plane A. In alternative fixation plates, there is a single row of fixator ports along the length of the plate. In yet other alternative fixation plates, the fixator ports can be mis-aligned in a variety of positions on the fixation plates.

The fixator ports have a neck face 164B that extends from external face 154B and narrows the fixator ports. This provides head surface 127B of enlarged head 132B of bone anchors 130B a surface to press against during an implanted mode. At the same time, the fixator ports are sufficiently large to pass the shank 133B of the bone anchors through where they can then be implanted in bone during an implanted mode. The neck face 164B profile in combination with the fixator ports can be configured to provide varying motion of the bone anchors. For example, the fixator ports can be configured to vary the amount of pivoting that occurs at axis E between the bone anchor and fixation plate.

Note that cervical fixation plate 102B includes three spaced pairs of fixator ports in this embodiment. As depicted in FIG. 23, fixation plate 102B spans across 2 cervical spine levels. In other embodiments, the fixation plate can comprise more or less pairs of fixator ports for spanning across more or less spine levels.

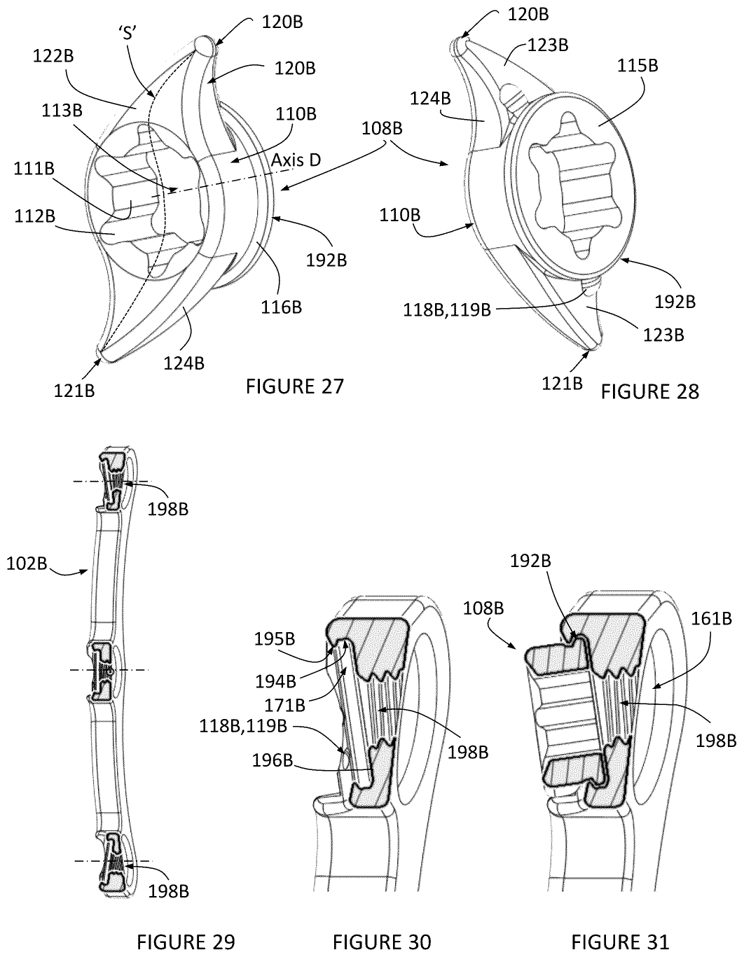

FIG. 24 also depicts an integrated blocking mechanism 106B of orthopedic fixation device 100B. Here, the integrated blocking mechanism comprises a pivotable blocking cam 108B that is inseparable from fixation plate 102B. As noted in FIG. 27-28, blocking cam 108B comprises a circular central body 110B. On a posterior side of the central body is a pivot flange 192B that radially encircles central body 110B and which extends to inward face 115B which in this embodiment is generally flat but can also be rounded. The opposing side of the pivot flange 192B is defined by pivot face 116B which faces the tabs.

Extending radially from an anterior portion of central body 110B are a pair of opposing tabs (first tab 120B, second tab 121B). In this embodiment, the opposing tabs form an ‘S’ shape. The tabs have a tab outer face 122B facing anterior in this case, and otherwise face away from the bone. A tab inner face 123B faces posterior in this case, and otherwise face toward the bone. A tab radial face 124B extends between the tab inner face and tab outer face which in this embodiment can be convex or concave to form the ‘S’, but can be generally straight in other embodiments. A detent face 119B on the blocking cam defines a detent 118B which can be in the form of, for example, a small boss or depression. A complementary detent 118B (FIG. 25) works to provide positions wherein the blocking cam remains in a blocked or unblocked position due to interference between the two opposing detents. In addition, the complementary detent can provide tactile feedback to the user to assist them in tactilely feeling an unblocked or blocked position.

Extending through a central axis (axis D) of the blocking cam 108B is a drive pocket 113B for receiving a drive tool to move the blocking cam 108B between a locked mode where the first tab or second tab interferes with a corresponding fixator port thereby preventing backout of a bone anchor, and between an unlocked mode where the first tab or second tab are positioned to not interfere with movement of a bone anchor through a fixator port. FIG. 24 depicts the blocking cams 108B in a locked mode, whereas if they were turned 90 degrees, they would assume an unlocked mode.

The fixation plate 102B comprises an anchor hole 198B (typically threaded) positioned between the paired first lateral fixator ports 161B and second lateral fixator port 162B. The anchor hole can be used by a driver to thread into the fixation plate before rotating the blocking cam thereby preventing the driver from slipping off. A seat face 196B is inset on the external face can be present to provide a surface for portions of the blocking cam 108B to rotate against. Adjacent to seat face 196B is a retainer lip 195B that defines a retainer groove 194B (FIG. 30,31) that receives pivot flange 192B therein. In this case, pivot flange 192B is seated in retainer groove 194B and the two are inseparable except through destruction of the fixation plate or blocking cam or both. Despite being inseparable, the blocking cam 108B can be rotated with respect to the fixation plate 102B between the locked mode (blocking enlarged head from back out) and unlocked mode (enlarged head unblocked from back out). The inseparable nature between blocking cam 108B and fixation plate 102B eliminates the need for assembly of parts and assures the parts will not disassemble once implanted.

The blocking cams described herein have an anchor portion that is larger than a restraint portion in a structural member where it resides. For example, in orthopedic fixation device 100, first tab 120 and second tab 121 (anchor portion) are larger than and blocked by cam windows 167 and cam port 172 (restraint portion) and thereby prevented from separation from intervertebral spacer 104 which serves as the structural member. As yet another example, in orthopedic fixation device 100B, pivot flange 192B (anchor portion) on blocking cam 108B is seated in retainer groove 194B. Pivot flange 192B is larger than the diameter opening defined by retainer lip 195B (restraint portion) thereby preventing the pivot flange from separation from fixation plate 102B.

Anatomical terms such as anterior and posterior are used with relation to placement of the intervertebral implant in a human body in an implanted mode.

It is noted that the terms “substantially” and “about” and “generally” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

The foregoing invention has been described in accordance with the relevant legal standards, thus the description is exemplary rather than limiting in nature. Variations and modifications to the disclosed embodiment may become apparent to those skilled in the art and fall within the scope of the invention.

1) An orthopedic fixation device operable for implantation in an intervertebral space in an implanted mode comprising:
an intervertebral spacer;
said intervertebral spacer comprising a boundary wall defining an external boundary of said intervertebral spacer;
said intervertebral spacer comprising a superior face on said boundary wall facing superiorly for engaging an endplate of a vertebral body;
said intervertebral spacer comprising an inferior face on said boundary wall facing inferiorly for engaging an endplate of a vertebral body;
said intervertebral spacer comprising an external face facing away from said intervertebral spacer and extending between said superior face and said inferior face on said boundary wall;
said intervertebral spacer comprising an internal face spaced inward from said external face on said boundary wall and extending between said superior face and said inferior face on said boundary wall;
at least a portion of said internal face defining a bone cavity extending between said superior face and said inferior face;
one or more fixator ports operable for housing at least a portion of a bone anchor therein;
said one or more fixator ports extending through said external face and extending toward one or more of said superior face and said inferior face;
a blocking cam;
said blocking cam comprising a round central body;
said blocking cam having a central drive pocket extending through said central body operable for receiving a tool to impart rotation on said blocking cam;
a pair of opposed tabs extending outward from said central body;
a cam port;
said cam port extending into said boundary wall through said external face;
said cam port at least partially defined by a rounded head capture face operable for seating a portion of said round central body therein;
a tab guide face;
said tab guide face extending from said head capture face;
said tab guide face defining a cam window extending between said cam port and adjacent said fixator port;
one of said opposed tabs at least partially housed in said cam window; and,
wherein said blocking cam is irremovable after manufacture from said intervertebral spacer without destruction of said blocking cam or said intervertebral spacer or both.

2) The orthopedic fixation device of claim 1 further comprising:
a cam port face at least partially defining said cam port;
said cam port face extending between said external face and said head capture face; and,
wherein said cam port face is of smaller diameter than said head capture face.

3) The orthopedic fixation device of claim 1 further comprising:
a second cam window;
said second cam window extending between said cam port and a second adjacent fixator port.

4) The orthopedic fixation device of claim 1 further comprising:
said fixator port comprising a reduced diameter neck face;
a bone anchor;
said bone anchor having an enlarged head;
wherein in said implanted mode, said enlarged head is positioned between said neck face and one of said opposed tabs.

5) The orthopedic fixation device of claim 1 further comprising:
a threaded control face;
said threaded control face extending between said head capture face and said internal face and operable for said threaded control face to engage a threaded portion of an insertion tool.

6) The orthopedic fixation device of claim 5 wherein said threaded control face has a smaller diameter than said drive pocket and is accessible through said drive pocket.

7) The orthopedic fixation device of claim 1 wherein entire said blocking cams are inset from said external face.

8) The orthopedic fixation device of claim 1 further comprising a plurality of one or the other or both of teeth and ridges extending from one or the other or both of said superior face and said inferior face.

9) The orthopedic fixation device of claim 1 wherein at least one cam port is positioned between two fixator ports.

10) The orthopedic fixation device of claim 1 wherein said intervertebral spacer is manufactured from a non-porous material.

11) The orthopedic fixation device of claim 1 wherein said intervertebral spacer comprises an open lattice structure.

12) The orthopedic fixation device of claim 1 wherein said blocking cams and said bone anchors are distanced internally from said external face in an implanted mode.

13) The orthopedic fixation device of claim 1 wherein said camports and said fixator ports extend through said external face on the anterior aspect.

14) The orthopedic fixation device of claim 1 wherein in a locked mode, tabs from a single blocking cam extend into two separate fixator ports.

15) The orthopedic fixation device of claim 1 wherein a portion of said fixator port extends through an internal face and at least one of said superior face and said inferior face.

16) The orthopedic fixation device of claim 1 further comprising two fixator ports that extend through said superior face.

17) The orthopedic fixation device of claim 1 wherein said drive pocket is partially defined by a plurality of inset drive faces for receiving torque from a tool.

18) The orthopedic fixation device of claim 1 wherein said head face and said head capture face are configured to have increased friction therebetween as said blocking cam approaches a locked mode.

19) The orthopedic fixation device of claim 1 further comprising:
a detent on said head face of said blocking cam;
a head dimple extending from said head capture face;
wherein interaction between said detent and said head dimple provide tactile feedback to the user to indicate one or the other or both of a locked mode and unlocked mode.

20) The orthopedic fixation device of claim 1 further comprising:
a plurality of bone anchors;
said bone anchors seated in said fixator ports;
one or more of said bone anchors extending through said superior face; and,
wherein said one or more of said bone anchors extend through said inferior face in an implanted mode.

21) An orthopedic fixation device in an implanted mode operable for fixation on a surface of a bone comprising:
a fixation plate;
said fixation plate comprising an internal face configured to face bone;
said fixation plate comprising an external face configured to face away from bone;
a bounding face encircling said fixation plate and extending between said internal face and said external face;
a plurality of fixator ports;
said fixator ports extending between said external face and said internal face and operable for seating a bone anchor therein;
said fixation plate having an integrated blocking mechanism;
said integrated blocking mechanism comprising a blocking cam positioned adjacent at least one fixator port;
said blocking cam comprising a circular central body;
one or more tabs extending outward from said central body;
a pivot flange on one end of said blocking cam encircling said central body; and,
wherein said blocking cam is irremovable from said fixation plate without destruction of said blocking cam or said bone plate or both.

22) The orthopedic fixation device of claim 21 further comprising a plurality of bone anchors operable for seating within said fixation ports to secure said fixation plate to bone.

23) The orthopedic fixation device of claim 21 wherein said fixator ports are aligned in pairs along said fixation plate.

24) The orthopedic fixation device of claim 21 wherein said blocking cam is positioned between a pair of said fixator ports.

25) The orthopedic fixation device of claim 21 wherein said blocking cam is switchable between a locked mode wherein said one or more tabs block removal of at least one of said bone anchors and an unlocked mode wherein said one or more tabs are clear from blocking removal of at least one of said bone anchors.

26) The orthopedic fixation device of claim 21 further comprising:
an anchor hole extending through said fixation plate;
said anchor hole being threaded; and,
wherein said anchor hole is aligned with said central body of said blocking mechanism.

27) The orthopedic fixation device of claim 21 further comprising a plate window spaced from a peripheral edge of said fixation plate and extending through said fixation plate.

28) The orthopedic fixation device of claim 21 further comprising:
a retainer groove formed in said fixation plate; and
wherein said pivot flange is seated in said retainer groove.

29) The orthopedic fixation device of claim 21 wherein said blocking cam comprises a drive pocket extending through said central body operable for driving said blocking cam between a locked mode and an unlocked mode.
